(19) United States
(12) Patent Application Publication
(10) Pub. No.: US 2026/0024632 A1
(43) Pub. Date: Jan. 22, 2026

(54) MONITORING AND ANALYSIS OF HEART SOUNDS AND SYMPTOMS FOR DETERMINATION OF RECOMMENDED ACTIONS

(71) Applicant: Becton, Dickinson and Company, Franklin Lakes, NJ (US)

(72) Inventors: Saman Parvaneh, Danver, MA (US); Carla Susana Cerqueira, Watchung, NJ (US); Ihsan J. Hasan, Lake Forest, CA (US); Zaniar Ardalan, Tucson, AZ (US); Cristhian M. Potes Blandon, Rancho Santa Margarita, CA (US); Robert Mohan Mac Carthy, Tustin, CA (US); Gokul Mohan Warrier, Irvine, CA (US); Venugopal Raghavendra Ghatikar, Irvine, CA (US)

(21) Appl. No.: 19/273,206

(22) Filed: Jul. 18, 2025

Related U.S. Application Data
(60) Provisional application No. 63/673,591, filed on Jul. 19, 2024.

(51) Int. Cl. G16H 10/60; G16H 40/67; G16H 50/20; G16H 70/20

(52) U.S. Cl. CPC G16H 10/60; G16H 40/67; G16H 50/20; G16H 70/20

(57) ABSTRACT

An example method of providing health condition information regarding a potential health condition of a subject is disclosed herein and can include collecting a sound of a heart of the subject; comparing the sound of the heart to a plurality of example sounds to detect an abnormality, for example, via a machine learning model; prompting, in response to the sound having an abnormality, the providing of symptom information regarding symptoms noticeable by the subject; determining, by a first machine learning model and depending upon the sound of the heart and the symptoms of the subject, the potential health condition; and providing the information to the subject depending upon the potential health condition.

### CROSS-REFERENCE TO RELATED APPLICATION(S)

This application claims the benefit of U.S. Provisional Application No. 63/673,591, filed Jul. 19, 2024, and entitled “MONITORING AND ANALYSIS OF HEART SOUNDS AND SYMPTOMS FOR DETERMINATION OF RECOMMENDED ACTIONS,” the disclosure of which is hereby incorporated by reference in its entirety.

### FIELD OF THE TECHNOLOGY

The disclosure relates generally to the determination of heart health and, in particular, to the monitoring of heart sounds and determining a potential health condition of the subject using the heart sound and symptoms experienced by the subject.

### BACKGROUND

Individuals with health conditions, such as heart disease and/or other heart conditions, are often unaware of negative symptoms and/or avoid seeking health care until the issue becomes dire due to the cost of healthcare services and/or other reasons. Additionally, individuals with health conditions may be unaware that they are experiencing health issues. In these situations, individuals may experience worsening of symptoms and delayed treatment, causing the individual's health to deteriorate and become life threatening. Thus, it may be advantageous to monitor an individual's health even when the individual is not aware he/she is experiencing symptoms and outside of a healthcare setting to determine if the individual is in need of medical care.

### SUMMARY

Potential health conditions of a subject/individual, such as a human or another mammal, can be determined by the disclosed systems and/or methods using at least one machine learning model and based on monitored heart sounds and symptoms experienced by the subject. The subject is solicited to provide symptoms based on a detection of abnormality from the heart sounds. In response to the detection of at least one abnormality, the machine learning model can determine the potential health conditions as well as information (including recommended actions, educational information, healthcare provider information, clinical trial information, etc.). The machine learning model can be altered/adjusted based upon the symptoms and the heart sounds to be more sensitive or more specific, or different machine learning models can be selected to tailor the determination/analysis to the heart sounds, symptoms, needs of the subject, and/or desires of healthcare providers. The systems and/or methods can be incorporated into and/or used in association with a wearable device (e.g., a smartwatch and/or a chest-worn device) and/or an electronic mobile device (e.g., a mobile phone), for example, through the use of a downloadable electronic mobile application.

An example method of providing health condition information regarding a potential health condition of a subject is disclosed herein and can include collecting a sound of a heart of the subject; comparing the sound of the heart to a plurality of example sounds to detect an abnormality; prompting, in response to the sound having an abnormality, the providing of symptom information regarding symptoms noticeable by the subject; determining, by a first machine learning model and depending upon the sound of the heart and the symptoms of the subject, the potential health condition(s); and providing the information to the subject depending upon the potential health condition(s).

An example health monitoring and analysis system for use in providing information regarding a potential health condition of a subject is disclosed herein and can include an abnormality detection module that includes a computer processor with the abnormality detection module being configured to receive at least one heart sound of the subject, compare the heart sound to a plurality of example heart sounds, and detect an abnormality; a symptom solicitation module configured to prompt, in response to the detection of an abnormality, the subject to provide at least one symptom noticeable by the subject; a machine learning model configured to determine, depending upon the heart sound and the at least one symptom, the potential health condition; and a notification module configured to provide information to the subject depending upon the potential health condition.

An example mobile application for use in providing information regarding a potential health condition of a subject is disclosed herein and can include a computer processor at least partially configured to receive a heart sound of the subject and perform executable software instructions to compare the heart sound to a plurality of example heart sounds; detect an abnormality in the heart sound from the comparison to the plurality of example heart sounds; prompt, in response to the heart sound having an abnormality, the subject to provide symptoms noticeable by the subject; and determine, depending upon the heart sound and the symptoms provided by the subject, the potential health condition. The example mobile application can also include a user interface configured to provide information to the subject regarding the potential health condition.

### BRIEF DESCRIPTION OF THE DRAWINGS

FIG. 1 is a schematic diagram of a health monitoring and analysis system.

FIG. 2 is a method flow chart describing an example process for determining and providing information regarding a potential health condition of a subject.

While the above-identified figures set forth one or more examples of the present disclosure, other examples are also contemplated, as noted in the discussion. In all cases, this disclosure presents examples by way of representation and not limitation. It should be understood that numerous other modifications can be devised by those skilled in the art, which fall within the scope and spirit of the principles of the disclosed systems and methods. The figures may not be drawn to scale, and applications and examples of the disclosed systems and methods may include features and components not specifically shown in the drawings.

### DETAILED DESCRIPTION

The disclosed example systems and methods provide information regarding a potential health condition to a subject. The information can include guidance that the subject should take regarding the potential health condition, information regarding healthcare providers, a digital map showing the locations of healthcare providers, educational details about the potential health conditions, information regarding clinical trial(s) that may be relevant to the subject, and a recommendation that the subject contacts a healthcare provider. The example systems and methods can monitor a subject's heart sound and evaluate the heart sound to detect any abnormalities. In response to the detection of an abnormality, the systems and methods can solicit/prompt the subject (or another associated with the subject) to provide any noticeable symptoms. The solicitation/prompting can, via a user interface, ask the subject questions regarding how the subject is feeling. For example, the systems and methods can include and/or function in conjunction with an electronic mobile device, such as on a mobile application, that includes a user interface. The systems and methods can include a machine learning model that determines the potential health condition(s) from the heart sound and the symptoms provided by the subject. Further, the systems and methods can then provide the subject information regarding the potential health condition using, for example, the user interface. The example systems and methods can perform other tasks and/or aid the subject in other ways, such as by alerting the subject as to the seriousness of the potential health condition, providing a reminder notice after a specified period of time to remind the subject to contact a healthcare provider, notifying the subject on any clinical trial deadlines, creating a report that includes information regarding the heart sound and potential health condition, providing the report to specified healthcare providers, and/or contacting emergency medical personnel. The disclosed example systems and methods can determine a potential health condition even if the subject provides an indication or states that he/she is experiencing no symptoms. Thus, the example systems and methods provide early notice to subjects of potential health conditions and encourage intervention to those subjects that are more likely to avoid seeking health care and/or may not know he/she is experiencing a health condition. These and other advantages are realized by reviewing the below disclosure. This disclosure uses the terms “heart sound” and “heart sounds” interchangeable as the heart sound(s) can be any length (e.g., one beat/cycle, multiple beats/cycles, and/or continuous) and/or have any characteristics.

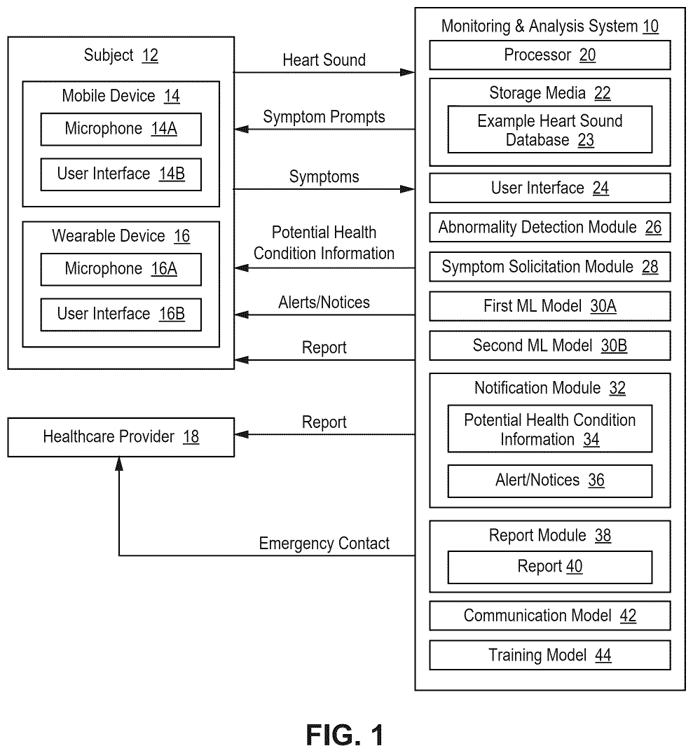

FIG. 1 is a schematic diagram of health monitoring and analysis system 10 (hereinafter also referred to as just “system 10”) for use with and/or in regards to subject 12. Further, system 10 can provide information to and/or receive information from healthcare provider 18. Subject 12 can have, include, and/or use mobile device 14 with microphone 14A and user interface 14B, and/or wearable device 16 with microphone 16A and user interface 16B. System 10 can include and/or function in conjunction with processor 20, storage media 22 (storing example heart sound database 23), user interface 24, abnormality detection module 26 (hereinafter also referred to as “detection module 26”), symptom solicitation module 28 (hereinafter also referred to as “solicitation module 28”), first machine learning module 30A (hereinafter referred to as the first “ML model” and/or as the first “sub-machine learning model”), second machine learning model 30B (hereinafter referred to as the second “ML model” and/or the second “sub-machine learning model”), notification module 32 configured to provide potential health condition information 34 and/or alerts/notices 36, report module 38 configured to generate/create report 40, communication module 42, and training module 44. While shown as being included within system 10, any of the components can be separate and distinct from system 10. For example, detection module 26, first ML model 30A, second ML model 30B, and training module 44 can be separate and distinct systems/components in communication with system 10. Additionally, while shown as separate systems/components, one example can be configured so system 10 is incorporated into and/or functions on/within mobile device 14 and/or wearable device 16 of subject 12, such as via an electronic mobile application.

FIG. 1 focuses on hardware components of monitoring and analysis system 10. FIG. 1 is provided as illustrative examples of a general hardware system for performing the capabilities discussed herein. The components presented in FIG. 1, particularly including models/modules 26, 28, 30A, 30B, 32, 38, 42, and 44 (and associated components) can be omitted or replaced with analogous hardware and/or software in different architectures without departing from the scope and spirit of the present disclosure.

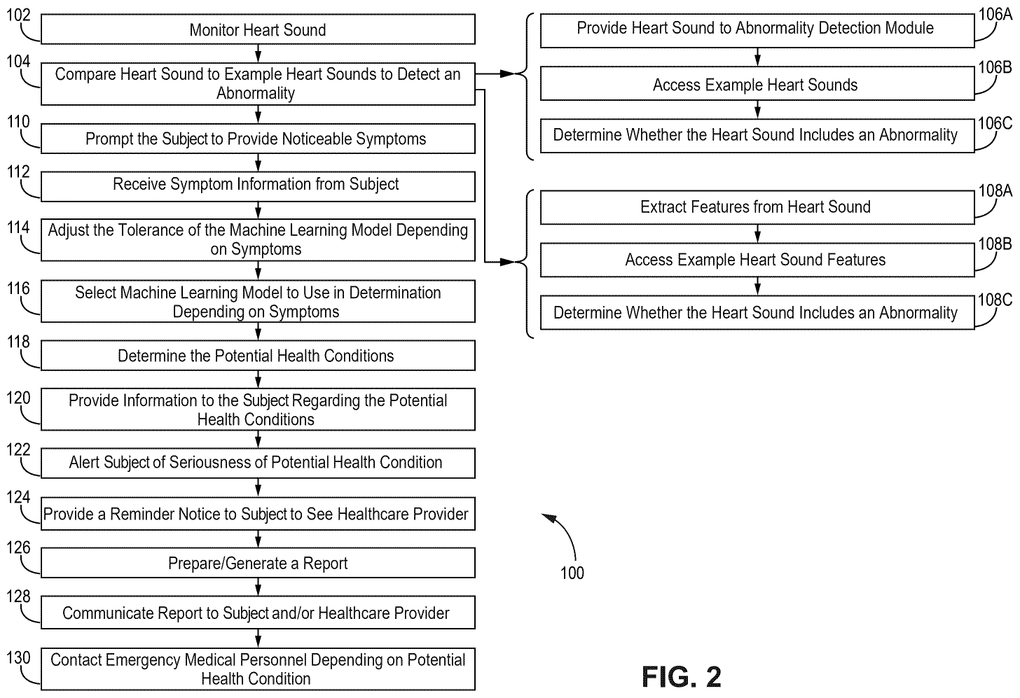

Monitoring and analysis system 10 (and process 100 described with regards to FIG. 2) can include other steps, components, models, modules, configurations, and/or features not expressly disclosed herein that are suitable for collecting/monitoring the heart sounds of subject 12, detecting any abnormalities in the heart sounds, soliciting and/or otherwise receiving symptoms experienced by subject 12, determining a potential health condition of subject 12, and/or providing information regarding the potential health condition to subject 12. For example, system 10 can include any number of digital/electronic storage media (e.g., storage media 22) for storing data, information, and/or executable instructions. System 10 can include any number of computer processors (e.g., processor 20) for performing tasks/instructions with regards to system 10 and/or process 100. Further, system 10 can allow for communication (e.g., communication module 42) via wired or wireless communication methods between components of system 10 and/or between other components, systems, subjects 12, mobile devices 14, wearable devices 16, healthcare providers 18, etc. distant from system 10. System 10 is described herein as including one or multiple “models” and/or “modules,” which can be any hardware and/or software for performing the tasks, functionality, and/or capabilities described herein. These “models” and/or “modules” can be instantiated in dedicated hardware and/or software, and/or can be defined functionally and use shared hardware and/or software.

Additionally, system 10 can be a discrete assembly or be formed by one or more components capable of individually or collectively implementing the functionalities described herein. In some examples, system 10 can be implemented as a plurality of discrete circuitry subassemblies. In some examples, one, multiple, or all components of system 10 can include and/or be implemented at least in part on a smartphone or tablet, among other options, such as mobile device 14 and/or wearable device 16. In some examples, one, multiple, or all components of system 10 can include and/or be implemented as downloadable software in the form of a mobile application. The mobile application can be implemented on a computing device, such as a personal computer, tablet, smartphone, and/or smartwatch, among other suitable devices, such as mobile device 14 and/or wearable device 16. One, multiple, or all components of system 10 can be considered to form a single computing device even when distributed across multiple component computing devices. System 10 can include a configuration in which one, multiple, or all of the functions described herein are performed by different components. System 10 can include various components for performing the above functions (as well as other functions described in this disclosure), such as processor 20, storage media 22, and/or user interface 14B, 16B, and/or 24.

System 10 can access, receive, and/or otherwise use information collected from subject 12, such as the heart sounds and/or symptoms of subject 12. The heart sounds can be collected using a variety of devices and/or via a variety of methods. In the example shown in FIG. 1, the heart sounds of subject 12 are collected via monitoring of subject 12 using microphone 14A of mobile device 14 and/or microphone 16A of wearable device 16. The heart sounds can be collected through listening to the heart for only a short time, for an extended period of time, and/or through continuous monitoring over the course of hours and/or days. In one example, subject 12 places mobile device 14 and/or wearable device 16 close to the heart of subject 12 so that microphone 14A and/or 16A can hear the heart sounds. System 10 may only need the heart sound to include one or a few beats/cycles of the heart, so the monitoring of the heart may not need to be for more than a few seconds or minutes. In this example, mobile device 14 is a mobile phone of subject 12 and/or wearable device 16 is a smartwatch and/or chest-worn device of subject 12. In another example, the heart sound is collected via other methods and/or devices not expressly disclosed herein. The heart sounds can be collected as initiated by subject 12 and/or another way, including automatically per a schedule. For example, the heart sounds can be collected automatically at the same time(s) each day as initiated and/or collected by mobile device 14 and/or wearable device 16. In one example, the heart sounds can be collected at 8 AM and 8 PM each day and/or on another schedule. If mobile device 14 and/or wearable device 16 is not in a position to be able to collect the heart sounds (e.g., is not within range to hear the heart sound), the heart sounds can be scheduled and/or attempted to be collected at another time. The heart sounds can be communicated to system 10 via any communication methods, including using communication module 42, using any wired and/or wireless communication, and/or using any communications capabilities of mobile device 14 and/or wearable device 16. In one example, system 10 is incorporated into (e.g., a mobile application on) mobile device 14 such that system 10 has access to microphone 14A to collect the heart sounds. Further, mobile device 14 can be in short-range wireless communication (e.g., Bluetooth) with wearable device 16 so that system 10 can receive the heart sounds from wearable device 16.

Symptoms of subject 12 can be collected and/or provided to system 10 using a variety of devices and/or a variety of methods. As described below with regards to symptom solicitation module 28 of system 10, subject 12 can be prompted (e.g., questions can be presented to subject 12) to provide any and/or all symptoms noticeable to subject 12. In one example, subject 12 can directly provide the symptoms via user interface 14B of mobile device 14 (e.g., a mobile application downloaded on mobile device 14), via user interface 16B of wearable device 16 (e.g., a mobile application downloaded on wearable device 16), via user interface 24 of system 10, and/or audibly via voice communication with a microphone/speaker and/or another smart/interactive device. In another example, symptoms of subject 12 can be provided by someone other than subject 12, such as the subject's healthcare provider, another caregiver, and/or a family member of subject 12. Additionally and/or alternatively, subject 12 can provide symptom information that in and of itself may not directly state/include all symptoms, but instead may need evaluation by system 10 to determine the symptoms experienced by subject 12 from the information provided by subject 12.

The symptoms can be provided by answering questions, selecting the symptoms from a list, entering the symptoms in individually (such as by using a keyboard/touch screen), audibly speaking the symptoms and/or other subject information into microphone 14A and/or 16A, and/or other methods. The symptoms can include anything experienced/noticeable by subject 12, such as shortness of breath, chest pain, chest tightness, feeling faint, feeling dizzy, heart palpitations, difficulty moving, swelling lower extremities, difficulty sleeping, and decline in activity level. Subject 12 can be solicited once or periodically to provide symptoms, and subject 12 can provide any changes to symptoms since the last time symptoms were provided to mobile device 14, wearable device 16, and/or system 10. The symptoms and/or information can be communicated to system 10 via any communication methods, including using communication module 42, using any wired and/or wireless communication, and/or using any communications capabilities of mobile device 14 and/or wearable device 16. In one example, system 10 is incorporated into (e.g., a mobile application on) mobile device 14 such that user interface 14B of mobile device 14 is the same as user interface 24 of system 10. Thus, the symptoms and/or information as provided via user interface 14B are provided directly to system 10. Further, mobile device 14 can be in short-range wireless communication (e.g., Bluetooth) with wearable device 16 so that system 10 can receive the symptoms of subject 12 from user interface 16B of wearable device 16.

System 10 can communicate with healthcare provider 18, which can be any person, clinic, hospital, care facility, research facility, emergency medical personnel, ambulance, company, etc. suitable for receiving information from system 10 regarding subject 12. The communication can include, for example, report 40 and/or emergency contact in response to the potential health condition revealing that subject 12 should receive immediate medical care. Healthcare provider 18 can have any communications capabilities to receive information from system 10 and potentially provide information to system 10.

As described above, system 10 can have any physical and/or digital location. In one example, system 10 is a stand-alone system. In another example, system 10 is incorporated into a mobile application (e.g., software) that is hosted/downloaded on mobile device 14 and/or wearable device 16 of subject 12. In a third example, system 10 is distant from subject 12 and can accommodate (e.g., be used by) multiple subjects 12 at the same time with information provided by each subject 12 via the internet or another wired/wireless communication method. System 10 is configured to accept, receive, and/or otherwise use the heart sound(s) and symptoms of subject 12 to determine a potential health condition experienced by subject 12 and provide information regarding that potential health condition to subject 12 and/or healthcare provider 18. System 10 can also, depending on the potential health condition, provide educational information (e.g., details about the potential health condition), recommended actions to subject 12 (e.g., guidance as to how subject 12 should proceed), information regarding healthcare providers 18, a digital map showing the location of healthcare providers 18, information regarding clinical trial(s) that may be relevant to subject 12, alerts to subject 12 regarding the seriousness of the potential health condition, reminder notices to subject 12 to see healthcare provider 18, potential deadlines regarding clinical trials, and/or other information. Additionally, system 10 can create/generate report 40 that includes, for example, the heart sounds of subject 12, a graph/image representative of the heart sounds, the symptoms as provided by subject 12, the potential health condition, and/or other information. System 10 can communicate report 40 to healthcare provider 18. System 10 can also contact emergency medical personnel, train one or all ML models 30A and/or 30B to improve performance, and/or have other capabilities.

System 10 (and/or the components of system 10) can include one or multiple computer/data processors 20 (also referred to herein as “processor 20”). In general, processor 20 can include any or more than one of a processor, a microprocessor, a controller, a digital signal processor (DSP), an application specific integrated circuit (ASIC), a field-programmable gate array (FPGA), or other equivalent discrete or integrated logic circuitry. Processor 20 can perform instructions stored within storage media 22 (or located elsewhere), and/or processor 20 can include memory such that processor 20 is able to store instructions and perform the functions described herein. Additionally, processor 20 can perform other computing processes described herein, such as the functions performed by any of the components of system 10 and/or any other systems/components shown in FIG. 1.

System 10 (and/or the components of system 10) can also include storage media 22. Storage media 22 is configured to store information (such as heart sounds, symptoms, and/or example heart sound database 23) and, in some examples, can be described as a computer-readable storage medium, media, and/or memory. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, storage media 22 is a temporary memory. As used herein, a temporary memory refers to a memory having a primary purpose that is not long-term storage. Storage media 22, in some examples, is described as volatile memory. As used herein, a volatile memory refers to a memory that does not maintain stored contents when power to storage media 22 is turned off. Examples of volatile memories can include random access memories (RAM), dynamic random access memories (DRAM), static random access memories (SRAM), and other forms of volatile memories. In some examples, the storage media/memory is used to store program instructions for execution by the processor. The memory, in one example, is used by software or applications running on system 10 to temporarily store information during program execution.

Storage media 22 can be configured to store larger amounts of information than volatile memory. Storage media 22 can further be configured for long-term storage of information. In some examples, storage media 22 includes non-volatile storage elements. Examples of such non-volatile storage elements can include, for example, magnetic hard discs, optical discs, floppy discs, flash memories, cloud storage media, or forms of electrically programmable memories (EPROM) or electrically erasable and programmable (EEPROM) memories. Additionally, storage media 22 can be digital/electronic storage in the “cloud” that is distant from the other components of system 10.

System 10 can also include user interface 24. User interface 24 can be an input and/or output device and enables an operator/user to control operation, modification, view of data, etc. of the heart sounds, symptoms, symptom prompts, potential health condition information 34, alerts/notices 36, reports 40, and/or the other information and/or systems/components within system 10 and/or in communication with system 10. For example, user interface 24 can be configured to receive inputs, such as heart sounds and/or symptoms, from subject 12 and/or provide information, such as potential health condition information 34, alerts/notices 36, and/or reports 40. User interface 24 can include one or more of a sound card, a video graphics card, a speaker, a display device (e.g., a liquid crystal display (LCD), a light emitting diode (LED) display, an organic light emitting diode (OLED) display, etc.), a touchscreen, a keyboard, a mouse, a joystick, and/or other type of device for facilitating input and/or output of information in a form understandable to users and/or machines. In one example, a user, operator, subject 12, and/or other individual can use user interface 24 to view and/or alter any of the information, heart sounds, symptom prompts, potential health condition information 34, alerts/notices 36, and/or reports 40 associated with system 10. In another configuration, user interface 14B of mobile device 14 and/or user interface 16B of wearable device 16 can include the same capabilities and/or functionalities as described above with regards to user interface 24. For example, user interfaces 14B, 16B, and/or 24 can be the same component(s) that work in conjunction with one another such that information provided to one user interface can be viewed, modified, etc. in another user interface and/or used by other components of system 10.

System 10 can include and/or work in conjunction with abnormality detection module 26. Detection module 26 can include and/or function in conjunction with any of the other components of system 10 (such as processor 20, storage media 22, and/or user interface 24). In one example, detection module 26 is part of and/or incorporated into one or both of ML models 30A and/or 30B such that the machine learning model performs the tasks disclosed herein as being performed by detection module 26. Further, detection module 26 can be on/within mobile device 14, wearable device 16, and/or a mobile application hosted/downloaded on any device/hardware. Detection module 26 can have other configurations, such as detection module 26 being and/or including an artificial intelligence model. Detection module 26 can access, receive, and/or otherwise use the heart sound(s) from subject 12 to detect an abnormality (and/or multiple abnormalities) in the heart of subject 12. Inversely, detection module 26 can evaluate the heart sound(s) and determine that no abnormality is present in the heart sound and/or that the heart sound is inconclusive as to the detection of an abnormality.

Detection module 26 can detect the abnormality by comparing the heart sound to example heart sounds. The example heart sounds can be, for example, stored and/or otherwise accessible in example heart sound database 23. In turn, example heart sound database 23 can be stored in, for example, storage media 22 of system 10. The example heart sounds can include many different heart sounds having any normal and/or abnormal sounds. For example, the example heart sounds can be of a heart that is experiencing one or multiple of the following: atrial fibrillation, heart murmurs, structural heart valve disease, precursors to cardiac arrest, a bicuspid aortic valve, and aortic valve stenosis. Further, the example heart sounds can include many different variations of similar normal and/or abnormal heart sounds. In one example, example heart sound database 23 includes hundreds of example heart sounds to which the heart sound from subject 12 is compared. Thus, detection module 26 can also be configured to access example heart sound database 23 to compare the heart sound from subject 12 to the example heart sounds.

Detection module 26 can be configured to extract heart sound features from the heart sounds and compare those features to example features in example heart sounds. The heart sound features can be, for example, the time and frequency of the heart sound, such as heart sound intervals (e.g., S1 intervals, S2 intervals, and systolic intervals), heart sound amplitudes (e.g., ratio of the mean absolute amplitude during systole to that during the S1 period in each heart beat), and frequency features (e.g., median power across different frequency bands). Detection module 26 can be configured to perform the comparison and detection manually as initiated and/or performed by a user/operator, and/or detection module 26 can be configured to perform the comparison and detection automatically in response to, for example, the reception of the heart sounds and/or in response to any other triggering event or instructions. Detection module 26 can be, for example, in communication with storage media 22 to access and/or receive information, such as the heart sounds and/or example heart sound database 23. In another example, detection module 26 can be a machine learning model that is trained on historical data with known labels (e.g., normal and abnormal heart sounds). The inputs to the machine learning model can be extracted heart sound features, the heart sound (e.g., raw data), and/or other inputs. Detection module 26 can be and/or use machine learning models that include support vector machines, ensemble classifiers, and/or deep learning models (e.g., convolutional neural networks and/or recurrence neural networks).

System 10 can include and/or work in conjunction with symptom solicitation module 28. Solicitation module 28 can include and/or function in conjunction with any of the other components of system 10 (such as processor 20, storage media 22, and/or user interface 24). In one example, solicitation module 28 is on/within mobile device 14, wearable device 16, and/or a mobile application hosted/downloaded on any device/hardware. In such a configuration, solicitation module 28 functions in conjunction with user interface 14B of mobile device 14 and/or user interface 16B of wearable device 16 to, for example, ask subject 12 questions regarding whether subject 12 is experiencing any symptoms. In this example and/or in other examples, user interface 24 of system 10 can be the same/incorporated into user interface 14B and/or user interface 16B. Solicitation module 28 can use a variety of devices, methods, etc. to solicit symptoms from subject 12 depending on whether an abnormality is detected in the heart sounds of subject 12 and/or depending on the type of abnormality detected in the heart sounds of subject 12. In a first example, solicitation module 28 (via user interface 14B, 16B, and/or 24) asks subject 12 one or multiple questions regarding how subject 12 is feeling (e.g., whether subject 12 is experiencing any symptoms). These questions can be “yes” or “no” questions, multiple choice questions, ask subjects 12 to select any noticeable symptoms from a list of symptoms, request that subject 12 manually type/enter any noticeable symptoms, and/or prompt subject 12 to provide symptoms using another format. In a second example, solicitation module 28 can audibly request subject 12 to provide symptoms, such as via a voice call that uses an automated system to which subject 12 can audibly provide symptoms. In a third example, solicitation module 28 can be configured to contact a healthcare provider, technician, etc. that is qualified to contact subject 12 and prompt subject 12 to provide symptoms. In other examples, solicitation module 28 can use other methods to prompt subject 12 to provide symptoms, such as providing an audible/oscillatory motion alert to draw the attention of subject 12 to the question/prompt on user interface 14B, 16B, and/or 24. While described herein as subject 12 providing symptoms, symptoms can be provided by anyone with knowledge of the symptoms of subject 12. For example, a family member, caregiver, healthcare provider, and/or others can provide symptoms of subject 12. Further, the symptoms and/or symptom information can be collected via other methods. Solicitation module 28 can also be configured to alter the prompts to subject 12 depending on the type of abnormality detected by detection module 26 and/or depending on the answer to prior questions regarding symptoms as provided by subject 12 to encourage subject 12 to provide information regarding all symptoms currently experienced by subject 12 as well as symptoms experienced by subject 12 in the past.

Solicitation module 28 can be configured to prompt subject 12 to provide symptoms manually as initiated and/or performed by a user/operator and/or prompt subject 12 automatically in response to, for example, the detection of at least one abnormality in the heart sounds of subject 12 and/or in response to any other triggering events/instructions. Further, solicitation module 28 can be configured to receive the symptoms (and/or associated information) as provided by subject 12 and, for example, store those symptoms in storage media 22, provide and/or allow access to the symptoms by ML models 30A and/or 30B, and/or take other actions with the information. Thus, solicitation module 28 can be in communication with storage media 22, other components of system 10, and/or subject 12 (e.g., mobile device 14 and/or wearable device 16).

System 10 can include and/or work in conjunction with first ML model 30A and/or second ML model 30B. First ML model 30A, second ML model 30B, any sub-machine learning models, and/or any other machine learning models described herein can be separate and distinct components, models, hardware, software, etc. and/or can be one machine learning model having and/or including similar components, elements, hardware, software, etc. For example, first ML model 30A and second ML model 30B can be the same machine learning model that is configured to perform the same or different instructions/determinations, and/or that is configured to perform the instructions/determinations using the same or different processes. In another example, first ML model 30A is a sub-machine learning model and second ML model 30B is another sub-machine learning model that are components of one machine learning model. When describing the characteristics, functionalities, and capabilities of ML models 30A and 30B in this disclosure, those characteristics, functionalities, and capabilities can also be present/performed by any of the machine learning models and/or sub-machine learning models set out in this disclosure.

ML models 30A and/or 30B (and any other ML models or sub-machine learning models described herein) can perform various techniques to create and/or adjust an algorithm (or multiple algorithms) or otherwise determine which inputs (e.g., the heart sounds and/or symptoms of subject 12) are most indicative of prediction of the outputs (e.g., the potential health conditions of subject 12). These techniques can include classification techniques (e.g., support vector machines, discriminant analysis, naïve bayes, nearest neighbor), regression techniques (e.g., linear regression, GLM, SVR, GPR, ensemble methods, decision trees, random decision forest, random forest, neural networks), clustering (e.g., K-means, K-medoids, fuzzy C-means, hierarchical, Gaussian mixture, neural networks, hidden Markov models), and/or other techniques, such as extreme gradient boosting (XGBoost), logistic regression, and time series forecasting. ML models 30A and/or 30B can determine and/or weight the importance of each input using coefficients that are increased and/or decreased to refine the accuracy of the prediction by ML models 30A and/or 30B. Other techniques and/or methods of training ML models 30A and/or 30B can be used by training module 44 to train ML models 30A and/or 30B.

ML models 30A and/or 30B can include and/or function in conjunction with any of the other components of system 10 (such as processor 20, storage media 22, and/or user interface 24). Further, ML models 30A and/or 30B can include, perform the tasks of, and/or work in conjunction with, for example, abnormality detection module 26 and/or training module 44. Further, ML models 30A and/or 30B can be on/within mobile device 14, wearable device 16, and/or a mobile application hosted/downloaded on any device/hardware. ML models 30A and/or 30B can have other configurations, such as ML models 30A and/or 30B being and/or including an artificial intelligence model. ML models 30A and/or 30B can be in communication with any of the components of system 10 to access, receive, and/or otherwise use the heart sound(s) from subject 12 and/or symptoms from subject 12 to determine the potential health conditions of subject 12.

As described above, ML models 30A and/or 30B can determine the potential health conditions by extracting features from the heart sounds and/or from the symptoms of subject 12 and/or through other processes, procedures, and/or techniques. The process of determining the potential health conditions can include the use of only one ML model 30A or 30B depending on the symptoms provided by subject 12. For example, first ML model 30A can be selected to determine the potential health condition(s) if the symptoms provided by subject 12 include that subject 12 is experiencing no noticeable symptoms. In this example, first ML model 30A is configured to have a tolerance that is focused on specificity (e.g., more focused/concerned with identifying a “healthy” subject as having no potential health conditions). In another example, second ML model 30B can be selected to determine the potential health condition(s) if the symptoms provided by subject 12 include at least one symptom that is noticeable by subject 12. In this example, second ML model 30B is configured to have a tolerance that is focused on sensitivity (e.g., is more focused/concerned with identifying an “unhealthy” subject as having at least one potential health condition). Further, the process of determining the potential health conditions can include adjusting a tolerance of one machine learning model to be more specific or more sensitive depending on the symptoms of subject 12 (e.g., ML models 30A and 30B are incorporated into one ML model and the tolerance of that one ML model is adjusted depending on the symptoms). In another example, the process of determining one or multiple potential health conditions by ML models 30A and/or 30B (and/or any of the other ML models or sub-machine learning models) can include determining whether the potential health condition and/or the heart sounds include the presence of a heart murmur in subject 12, determining whether the heart murmur is normal or abnormal, and determining a severity of the potential health condition. Each of these “steps” can be performed by one ML model, by multiple ML models, and/or by different components of system 10 (e.g., each step is performed by a separate ML model trained for that particular task/purpose).

ML models 30A and/or 30B can be configured to determine the potential health conditions manually as initiated and/or performed by a user/operator, and/or ML models 30A and/or 30B can be configured to determine the potential health conditions automatically in response to, for example, the reception of and/or access to the heart sounds and symptoms and/or in response to any other triggering event/instructions. ML models 30A and/or 30B can be, for example, in communication with storage media 22 to access and/or receive information, such as the heart sounds and/or symptoms of subject 12.

System 10 can include and/or work in conjunction with notification module 32, and notification module 32 can include and/or function in conjunction with any of the other components of system 10 (such as processor 20, storage media 22, and/or user interfaces 14B (of mobile device 14), 16B (of wearable device 16), and/or 24). In one example, notification module 32 is on/within mobile device 14, wearable device 16, and/or a mobile application hosted/downloaded on any device/hardware. Notification module 32 can be configured to provide information/notices to subject 12, such as potential health condition information 34 and/or alerts/notices 36. For example, notification module 32 can be configured to provide information regarding the potential health condition to subject 12 depending on the determination of the potential health condition by ML models 30A and/or 30B. The information provided to and/or made accessible to subject 12 by notification module 32 (and/or via user interfaces 14B, 16B, and/or 24) can include at least one of the following: information regarding healthcare providers 18, a digital map showing a location of at least one healthcare provider 18, details about the potential health condition (such as educational information), information regarding clinical trials relevant to subject 12, and/or a recommendation that the subject contact healthcare provider 18. The information provided by and/or made accessible by notification module 32 can be in any format and can be communicated via a variety of methods. Further, notification module 32 (and/or via user interfaces 14B, 16B, and/or 24) can alert subject 12 of the seriousness of the potential health condition and/or can provide a reminder notice to subject 12 to see healthcare provider 18 once and/or periodically after a specified amount of time has passed. For example, notification module 32 can alert subject 12 that he/she should seek the assistance of healthcare provider 18 as soon as possible depending on the potential health condition. In another example, notification module 32 can provide a reminder notice inquiring about whether subject 12 has been to healthcare provider 18 since the determination of the potential health condition. In a third example, notification module 32 can provide to (and/or allow access to) subject 12 any other information, including a notice that abnormality detection module 26 has detected an abnormality, a notice that the determination of the potential health condition returned that no potential health condition is present and/or the determination was inconclusive, and/or that report 40 has been generated and/or provided/made accessible to healthcare provider 18. Notification module 32 can include audible alerts/notifications, textual and/or visual alerts/notifications, and/or any other type of alerts/notifications configured to provide/make accessible any information to subject 12.

Notification module 32 can be configured to provide and/or make accessible potential health condition information 34, alerts/notification 36, and/or other information manually as initiated and/or performed by a user/operator, and/or provide/allow access to information automatically in response to, for example, the determination of potential health conditions and/or in response to any other triggering events/instructions. Further, notification module 32 can be configured to select, retrieve, and/or otherwise gain access to the information as located in storage media 22 and/or at another location, such as in the cloud as accessed via the internet. Thus, notification module 32 can be in communication with any components of system 10 and/or any sources of information regarding subject 12, the potential health conditions, healthcare providers 18, and/or other information.

System 10 can include and/or work in conjunction with report module 38, and report module 38 can include and/or function in conjunction with any of the other components of system 10 (such as processor 20, storage media 22, and/or user interfaces 14B (of mobile device 14), 16B (of wearable device 16), and/or 24). Report module 38 can be configured to prepare, create, and/or otherwise generate report 40 regarding subject 12 that can include identification information of subject 12, the heart sounds, descriptions of the heart sounds, images representative of the heart sounds, the symptoms, the potential health conditions, and/or any other information regarding subject 12 and/or the analysis of subject 12 for potential health conditions. Report 40 can include information in any format and/or can be in multiple formats. For example, report 40 can include an audio file of the heart sounds as well as a PDF file format that includes subject 12 information and information regarding the potential health conditions. Report module 38 can be configured to generate report 40 manually as initiated and/or performed by a user/operator (e.g., subject 12), and/or generate report 40 automatically in response to, for example, the determination of potential health conditions and/or in response to any other triggering events/instructions. For example, report module 38 can be instructed to generate report 40 by subject 12 and/or in response to subject 12 making an appointment seeking assistance from healthcare provider 18. Once generated, notification module 32 can be used to provide and/or otherwise make report 40 available to subject 12. Report module 38 can be configured to access the information that is to be included in report 40 from any components of system 10, and can be configured to provide report 40 to, for example, storage media 22 within which report 40 can be saved.

System 10 can include and/or work in conjunction with communication module 42, and communication module 42 can include and/or function in conjunction with any of the other components of system 10 (such as processor 20, storage media 22, and/or user interfaces 14B (of mobile device 14), 16B (of wearable device 16), and/or 24). Communication module 42 can be configured to provide and/or make accessible information within system 10 to components, systems, people, etc. outside (i.e., separate from) system 10. For example, communication module 42 can be configured to provide report 40 to healthcare provide 18. In another example, communication module 42 is configured to, in response to the determination that the potential health condition is serious and requires immediate medical attention, contact emergency personnel, such as healthcare provider 18. Communication module 42 can have other functionalities and/or capabilities, such as providing communication between system 10, mobile device 14, and/or wearable device 16. Communication module 42 can be configured to provide report 40 and/or contact a relative, friend, and/or other individual close to subject 12, professional caregiver, and/or emergency medical personnel manually as initiated and/or performed by a user/operator (e.g., subject 12) and/or automatically in response to, for example, the generation of report 40, determination of potential health conditions, and/or any other triggering events/instructions. Communication module 42 can have access to one, multiple, or all components of system 10 (e.g., storage media 22) to access the information that is to be communicated to healthcare provider 18 and/or to any other desired parties/components. In one example, communication module 42 has access to the telephone function/application on mobile device 14 to be able to contact a relative, friend, and/or other individual close to subject 12, professional caregiver, and/or emergency medical personnel. In another example, communication module 42 has access to cellular data and/or other wireless communication capabilities of mobile device 14 and/or wearable device 16 to send and receive information.

System 10 can include and/or work in conjunction with training module 44. Training module 44 can include and/or function in conjunction with any of the other components of system 10 (such as processor 20, storage media 22, and/or user interface 24). In one example, training module 44 is part of and/or incorporated into one or both of ML models 30A and/or 30B. Training module 44 is configured to train ML models 30A and/or 30B using, for example, the heart sounds of subjects 12, the symptoms of subjects 12, and/or the potential health conditions (and associated information). Training module 44 can include, be in communication with, and/or use any of the components of system 10. The heart sounds, symptoms, and potential health conditions can be used by ML models 30A and/or 30B as test inputs and outputs, respectively. Training module 44 can be configured to train ML models 30A and/or 30B with data/information from one subject 12 for further use of the trained ML models 30A and/or 30B on that subject 12 and/or for further use of the trained ML models 30A and/or 30B on other subjects. Training module 44 can be in communication with any of the components of system 10 to access, receive, and/or otherwise use data/information to train ML models 30A and/or 30B.

System 10 can determine the potential health condition(s) from the heart sound(s) and the symptoms provided by subject 12. Further, system 10 can then provide subject 12 information regarding the potential health condition using, for example, user interface 14B of mobile device 14, user interface 16B of wearable device 16, and/or user interface 24. The example system 10 can perform other tasks and/or aid subject 12 in other ways, such as by alerting subject 12 as to the seriousness of the potential health condition, provide a reminder notice after a specified period of time to remind subject 12 to contact healthcare provider 18, create report 40 that includes information regarding the heart sounds and potential health condition, provide report 40 to specified healthcare providers 18, and/or contact a relative, friend, and/or other individual close to subject 12, professional caregiver, and/or emergency medical personnel. The disclosed example system 10 can determine a potential health condition even if subject 12 provides/states that he/she is experiencing no symptoms. Thus, the example system 10 provides early notice to subjects 12 of potential health conditions and encourage intervention to those subjects 12 that are more likely to avoid seeking health care and/or that are unaware he/she is experiencing a health condition.

FIG. 2 is a method flow chart describing example process 100 for determining potential health conditions and providing information regarding the potential health conditions to a subject (e.g., subject 12). While process 100 is described herein as being used with regards to monitoring and analysis system 10 (and/or by/on mobile device 14 and/or wearable device 16), process 100 can be performed by any system(s) having any components, capabilities, configurations, and/or functionalities suitable for performing process 100. Additionally, process 100 can include other steps not expressly disclosed herein and/or can include performing the disclosed steps in any order and/or multiple times as is desired and/or necessary to determine the potential health conditions and/or provide information regarding the potential health conditions. Moreover, not all steps of process 100 must be performed, and process 100 can be performed partially or entirely in a digital environment by and/or within the systems/components set out in this disclosure, such as monitoring and analysis system 10, mobile device 14, wearable device 16, and/or other systems/components.

Process 100 can include step 102, which is to monitor, record, and/or otherwise collect the heart sound(s) of subject 12. The heart sounds can be collected using a variety of devices and/or methods. In one example, step 102 is performed by monitoring subject 12 using microphone 14A of mobile device 14 and/or microphone 16A of wearable device 16. Step 102 can be performed to collect the heart sounds through listening to the heart for only a short time, for an extended period of time, and/or through continuous monitoring over the course of hours and/or days. In one example, step 102 is performed by subject 12 placing mobile device 14 (e.g., a mobile telephone) and/or wearable device 16 (e.g., a smartwatch and/or chest-worn device) close to the heart of subject 12 so that microphone 14A and/or 16A can hear the heart sounds. Step 102 can include collecting only one or a few beats/cycles of the heart, so step 102 may only need to be performed for a few seconds or minutes. In another example, step 102 is performed via other methods and/or using other devices not expressly disclosed herein. Step 102 can be performed and/or initiated manually by subject 12 (or another individual/system) and/or another way, including automatically and/or once or periodically on a schedule. For example, step 102 can be performed automatically at the same time(s) each day as initiated and/or collected by mobile device 14 and/or wearable device 16. In this example, step 102 can be performed once at 8 AM and another time at 8 PM each day and/or on another schedule. In mobile device 14 and/or wearable device 16 is not in position to be able to collect the heart sounds (e.g., is not within range to hear the heart sounds), step 102 can be scheduled and/or attempted at another time. Step 102 can also include communicating the heart sounds to other components of system 10 and/or to other systems. The communication can be via any wired and/or wireless communication, such as via the communication capabilities of mobile device 14 and/or wearable device 16. Step 102 can be performed once, multiple times, and/or continuously as is desired and/or necessary to collect the heart sound(s) for further analysis by process 100.

Process 100 can include step 104, which is to compare the heart sound to example heart sounds to detect an abnormality. Step 104 can include multiple sub-steps 106A-106C and/or 108A-108C. Step 104 can be performed by, for example, abnormality detection module 26 of system 10. Additionally and/or alternatively, step 104 can be performed by any components of system 10 and/or any systems capable of detecting at least one abnormality from the heart sounds of subject 12. For example, step 104 can be performed by an artificial intelligence model, a machine learning model, and/or another model configured to analyze the heart sounds and detect an abnormality in the heart sounds.

Step 104 can include sub-step 106A, which is to provide/make accessible the heart sounds to abnormality detection module 26 and/or another model/module for detecting an abnormality. Detection module 26, in sub-step 106A can access, receive, and/or otherwise use the heart sound(s) from subject 12. Sub-step 106A can be performed via any communication methods, such as wired and/or wireless communication. Additionally and/or alternatively, sub-step 106A can include providing/saving the heart sounds in storage media 22 (and/or at another location) and then accessing/retrieving the heart sounds by detection module 26 from that location.

Next, sub-step 106B includes accessing example heart sounds from, for example, example heart sound database 23 in storage media 22. Sub-step 106B can include accessing, receiving, and/or otherwise using one, multiple, and/or all example heart sounds in the analysis of the heart sound(s) from subject 12. The example heart sounds in example heart sound database 23 can be labeled and/or otherwise organized easy access and/or use.

Finally, step 104 can include sub-step 106C, which is determining whether the heart sound has an abnormality that warrants further analysis in process 100. Sub-step 106C can include comparing the heart sound to example heart sounds to detect whether the heart sounds include at least one abnormality. The abnormality can be indicative of at least one of the following: atrial fibrillation, heart murmurs, structural heart valve disease, precursors to cardiac arrest, a bicuspid aortic valve, and/or aortic valve stenosis. The determination in sub-step 106C can be a comparison of the audio of the heart sounds to the audio recordings of one or multiple example heart sounds. Additionally and/or alternatively, as set out in sub-steps 108A-108C, the determination in sub-step 106C can include extracting features from the heart sounds and comparing at least one of those features to example features associated with the example heart sounds to detect whether the heart sounds include at least one abnormality. Sub-step 106C can also include determining that the heart sound does not include an abnormality and/or that the heart sound is inconclusive as to the detection of an abnormality.

Additionally and/or alternatively to sub-steps 106A-106C, step 104 can include sub-steps 108A-108C. Sub-step 108A can include extracting features from the heart sound(s) of subject 12. The heart sound can include particular features/characteristics that can be used to determine if the heart sound is normal or includes at least one abnormality. The heart sound features can be, for example, the time and frequency of the heart sound, such as heart sound intervals (e.g., S1 intervals, S2 intervals, and systolic intervals), heart sound amplitudes (e.g., ratio of the mean absolute amplitude during systole to that during the S1 period in each heart beat), and frequency features (e.g., median power across different frequency bands). These features can be recorded, denoted, saved, etc. in sub-step 108A for further analysis.

Next, step 104 can include sub-step 108B, which is to access the example heart sound features from, for example, example heart sound database 23 in storage media 22. Sub-step 108B can include accessing, receiving, and/or otherwise using one, multiple, and/or all example heart sound features in the analysis of the heart sound(s) from subject 12. The example heart sound features can be the same type of features as those of the heart sound(s) and/or can be different and/or additional features to which the features of the heart sounds from subject 12 are compared. In this example, one, multiple, or all features of the heart sounds can be compared to the example heart sound features in sub-step 108C.

Finally, step 104 can include sub-step 108C, which is determining whether the heart sound has an abnormality that warrants further analysis in process 100. Sub-step 108C can include comparing features of the heart sound to example features of the example heart sounds to detect at least one abnormality in the heart sound. This comparison can be performed similarly to sub-step 106C as detailed above, but sub-step 108C can include comparing one, multiple, and/or all features of the heart sound to the example features of the example heart sound to detect whether the heart sound includes at least one abnormality. Sub-step 108C can also include determining that the heart sound does not include an abnormality and/or that the heart sound is inconclusive as to the detection of an abnormality. Additionally and/or alternatively, sub-step 108C can include determining the heart sound features that are most indicative of the presence of an abnormality (and, inversely, the presence of a normal heart sound).

Step 104 can include providing a notice to other components of system 10 and/or other systems that an abnormality has been detected so that process 100 continues to determine the potential health condition(s) and, inversely, that the heart sound does not include an abnormality. Further, step 104 can include providing the results of the determination (whether the heart sound includes an abnormality or not) to subject 12, for example, via any of user interfaces 14B, 16B, and/or 24. Step 104 can also include saving the determination to, for example, storage media 22 along with the heart sound (and/or information associated with the heart sound).

Step 104 (and/or sub-steps 106A-106C and/or 108A-108C) can include other steps, sub-steps, and/or methods for detecting whether the heart sound includes at least one abnormality, including using an artificial intelligence model and/or one or multiple machine-learning models. For example, step 104 can include determining which features/sounds are more indicative of the presence/detection of an abnormality and adjusting the determination/comparison to more accurately detect at least one abnormality. Step 104 can include other methods, sub-steps, etc. to increase the accuracy of the detection of at least one abnormality from the heart sound(s) of subject 12. Additionally, step 104 can be performed and/or initiated manually such that at least one abnormality is detected by a user/operator. Moreover, step 104 can be performed automatically in response to, for example, the reception of a heart sound and/or any other triggering events/instructions. Step 104 can be performed once, multiple times, and/or continuously as the heart sound(s) are recorded, collected, and/or otherwise used in step 104. Step 104 can further include communicating that at least one abnormality has been detected in the heart sound to subject 12 and/or to other components/systems to continue performance of process 100 to determine the potential health condition, such as mobile device 14, wearable device 16, symptom solicitation module 28, ML models 30A and/or 30B, and/or other components.

Next, in response to the detection of at least one abnormality in step 104, process 100 can have step 110. Step 110 can include prompting subject 12 to provide any symptoms experienced by subject 12. Step 110 can be performed by, for example, symptom solicitation module 28 as detailed with regards to system 10. Additionally and/or alternatively, step 110 can be performed and/or aided by any components of system 10 and/or any systems capable of prompting subject 12 for symptoms. For example, step 110 can be performed via and/or in conjunction with user interface 14B of mobile device 14, user interface 16B of wearable device 16, and/or user interface 24 of system 10 to ask subject 12 questions regarding whether subject 12 is experiencing any symptoms. In this example and/or in other examples, user interface 24 of system 10 can be the same/incorporated into user interface 14B and/or user interface 16B. Step 110 can include using a variety of devices, methods, etc. to solicit symptoms from subject 12 depending on whether an abnormality is detected in the heart sounds of subject 12 and/or depending on the type of abnormality detected in the heart sounds of subject 12 in step 104. In a first example, step 110 includes asking subject 12 one or multiple questions regarding how subject 12 is feeling (e.g., whether subject 12 is experiencing any symptoms). These questions can be “yes” or “no” questions, multiple choice questions, ask subjects 12 to select any noticeable symptoms from a list of symptoms, request that subject 12 manually type/enter any noticeable symptoms, and/or prompt subject 12 to provide symptoms using another format. In a second example, step 110 can include an audible request to subject 12 to provide symptoms, such as via a voice call that uses an automated system to which subject 12 can audibly provide symptoms. In a third example, step 110 can include contacting a healthcare provider, technician, etc. that is qualified to, in turn, contact subject 12 and prompt subject 12 to provide symptoms. In other examples, step 110 can include other steps to prompt subject 12 to provide symptoms, such as providing an audible/oscillatory motion alert to draw the attention of subject 12 to the question/prompt on user interface 14B, 16B, and/or 24. Step 110 can also include altering the prompts to subject 12 depending on the type of abnormality detected in step 104 and/or depending on the answer to prior questions regarding symptoms as provided by subject 12 to encourage subject 12 to provide information regarding all symptoms currently experienced by subject 12 as well as symptoms experienced by subject 12 in the past. Additionally, step 110 can be performed and/or initiated manually to prompt subject 12 to provide symptoms, and/or step 110 can be performed automatically such that subject 12 is prompted to provide symptoms in response to, for example, the detection of an abnormality and/or any other triggering events/instructions.

After step 110, process 100 can include step 112, which is receiving symptom information from subject 12. In response to the solicitation/prompting in step 110, subjects 12 can provide symptoms via, for example, user interface 14B of mobile device 14, user interface 16B of wearable device 16, and/or user interface 24. The symptoms can be provided, received, accessed, and/or otherwise used in any format suitable for use in determining the potential health conditions. Step 112 can be performed via any communication methods, such as wired and/or wireless communication (e.g., wireless communication from mobile device 14 and/or wearable device 16). Further, step 112 can include storing the symptoms as provided by subject 12 in, for example, storage media 22 and/or at any other location, such as in cloud storage. The symptoms, as provided by subject 12, can include anything that subject 12 is experiencing, such as shortness of breath, chest pain, chest tightness, feeling faint, feeling dizzy, heart palpitations, difficulty moving, swelling lower extremities, difficulty sleeping, and decline in activity level. The symptoms can be provided in step 112 in any format and/or method, including in a digital format, audibly, and/or in a physical document.

Process 100 can optionally include steps 114 and/or 116 to tailor the machine learning model to better suit the needs of subject 12 and/or the desires of a user/operator (as described above with regards to ML models 30A and/or 30B). Process 100 can include performing neither steps 114 and 116, only one of steps 114 and 116, or both steps 114 and 116.

Step 114 includes adjusting the tolerance of the machine learning model (e.g., ML models 30A and/or 30B) depending on the symptoms of subject 12. Step 114 can be performed by, for example, ML models 30A and/or 30B, any components of system 10, and/or any other systems. Step 114 can include adjusting the tolerance of the machine learning model, in response to the symptoms provided by subject 12 including that subject 12 is experiencing no noticeable symptoms (i.e., subject is asymptomatic), to focus on specificity (e.g., more focused/concerned with identifying a “healthy” subject as having no potential health condition). Inversely, step 114 can also include adjusting the tolerance of the machine learning model, in response to the symptoms provided by subject 12 including that subject is experiencing at least one noticeable symptom, to focus on sensitivity (e.g., is more focused/concerned with identifying an “unhealthy” subject as having at least one potential health condition). Further, step 114 can include adjusting a tolerance of one machine learning model to be more specific or more sensitive depending on the symptoms of subject 12. Step 114 can include adjusting the tolerance and/or other aspects of the machine learning model to focus on other factors, features, symptoms, etc. Step 114 can be performed and/or initiated manually such that the adjustment of the machine learning model is by a user/operator. Moreover, step 114 can be performed automatically such that the machine learning model is adjusted in response to, for example, the reception of symptoms from subject 12, the reception of the heart sounds/abnormality detection, and/or any other triggering events/instructions. In this example, the tolerance to which the machine learning model is adjusted can be preset and/or predetermined depending on any symptoms and/or combinations of symptoms of subject 12 such that the adjustment is performed automatically depending on the heart sounds and/or the symptoms.

Step 116 can include selecting a machine learning model to use in determining the potential health conditions. The selection in step 116 can be dependent upon, for example, the symptoms of subject 12. Step 116 can be similar to step 114 except that, instead of adjusting the tolerance of one machine learning model, step 116 selects from multiple different machine learning models having, for example, differing tolerances/characteristics. For example, first ML model 30A can be selected to determine the potential health condition(s) if the symptoms provided by subject 12 include that subject 12 is experiencing no noticeable symptoms. In this example, first ML model 30A is configured to have a tolerance that is focused on specificity (e.g., more focused/concerned with identifying a “healthy” subject as having no potential health condition). In another example, second ML model 30B can be selected to determine the potential health condition(s) if the symptoms provided by subject 12 include at least one symptom that is noticeable by subject 12. In this example, second ML model 30B is configured to have a tolerance that is focused on sensitivity (e.g., is more focused/concerned with identifying an “unhealthy” subject as having at least one potential health condition). In a third example, step 116 includes selecting a third, fourth, fifth, etc. ML model to focus on other factors, features, symptoms, etc. and/or depending on the types of symptoms of subject 12 (as opposed to selecting the ML model based on whether subject 12 is symptomatic or asymptomatic). As with step 114, step 116 can be performed and/or initiated manually such that the selection of the machine learning model is by a user/operator. Moreover, step 116 can be performed automatically such that the machine learning model is selected in response to, for example, the reception of symptoms from subject 12, the reception of the heart sounds/abnormality detection, and/or any other triggering events/instructions. In this example, the selection of the ML model can be predetermined depending on any symptoms and/or combinations of symptoms of subject 12 such that the selection is performed automatically depending on the heart sounds and/or the symptoms.

Process 100 can include step 118, which is determining the potential health condition(s) in subject 12 depending on the heart sound(s) and symptom(s) of subject 12. Step 118 can be performed by, for example, first ML model 30A, second ML model 30B, any sub-machine learning models, and/or any other machine learning models described herein (which can be present and/or used in conjunction with system 10, mobile device 14, wearable device 16, and/or other systems, software, hardware, etc.). As described above with regards to ML models 30A and/or 30B, the determination of the potential health conditions in step 118 can use various techniques to create and/or adjust an algorithm (or multiple algorithms) or otherwise determine which inputs (e.g., the heart sounds and/or symptoms of subject 12) are most indicative of predicting the outputs (e.g., the potential health conditions of subject 12). Please refer to the discussion above with regards to ML models 30A and 30B for further information regarding these techniques able to be used in step 118.

The potential health conditions determined in step 118 can be, for example, atrial fibrillation, heart murmurs, structural heart valve disease, precursors to cardiac arrest, a bicuspid aortic valve, and aortic valve stenosis present/experienced by subject 12. Step 118 can further include accessing, receiving, and/or otherwise using the heart sound(s) and symptom(s) as previously recorded and/or provided/collected in process 100. Step 118 can determine the potential health conditions by extracting features from the heart sounds and/or from the symptoms of subject 12 and/or through other processes, procedures, and/or techniques. Step 118 can be performed by one or multiple machine learning models as, for example, determined in steps 114 and/or 116. Step 118 can determine the potential health conditions manually as initiated and/or performed by a user/operator, and/or step 118 can be configured to determine the potential health conditions automatically in response to, for example, the reception of and/or access to the heart sounds and symptoms and/or in response to any other triggering event/instructions. Step 118 can include, for example, communicating with storage media 22 to access and/or receive information, such as the heart sound(s) and symptoms, and to save information, such as the potential health conditions.

Process 100 can include one, multiple, or all of steps 120-124 for providing information, alerts, notices, etc. to subject 12 and/or to others associated with subject 12 regarding the potential health conditions and/or follow up after the determination of the potential health conditions. Steps 120-124 can be performed by, for example, notification module 32 and/or via user interfaces 14B, 16B, and/or 24. Additionally and/or alternatively, steps 120-124 can be performed by any components of system 10 and/or any systems capable of providing information, alerts, notices, etc. to subject 12. Steps 120-124 can include providing information, alerts, notices, etc. audibly, textually and/or visually, and/or another method/process configured to provide/make accessible information to subject 12. In one example, the information, alerts, notices, etc. are provided to someone associated with subject 12 (such as a family member, caregiver, and/or healthcare provider 18) and is then conveyed to subject 12.

Step 120 can include providing information regarding the potential health condition to subject 12. The information provided to subject 12 in step 120 can include, for example, the potential health condition, information regarding healthcare providers 18, a digital map showing a location of at least one healthcare provider 18, details about the potential health condition (such as educational information), information regarding clinical trial(s) that may be relevant to subject 12, and/or a recommendation that the subject contact healthcare provider 18. The information provided by and/or made accessible in step 120 can be in any format and can be communicated via a variety of methods described above.

Step 122 can include alerting subject 12 of the seriousness of the potential health condition determined in step 118. The alert can be prompted by the determination of the potential health condition. Step 122 can include accessing a list of potential health conditions that warrant an alert to subject 12, and step 122 can further include comparing the potential condition as determined in step 118 to the list of potential health conditions to determine if an alert is necessary. The alert of step 122 can advise subject 12 that he/she should seek the assistance of healthcare provider 18 as soon as possible depending on the potential health condition. The alert can be provided by and/or made accessible in step 122 can be in any format and can be communicated via a variety of methods described above.

Step 124 can include providing a reminder notice inquiring about whether subject 12 has been to healthcare provider 18 since the determination of the potential health condition. For example, step 124 can send a reminder notice to subject 12 two weeks after the initial determination of the potential health condition inquiring as to whether subject 12 has been to healthcare provider 18 and/or has made an appointment to see healthcare provider 18. Further, step 124 can provide to (and/or allow access to) subject 12 any other information, including a notice that an abnormality was detected in step 104, a notice that the determination of the potential health condition in step 118 returned that no potential health condition is present and/or the determination was inconclusive, and/or that report 40 has been generated in step 126 and/or provided/made accessible to healthcare provider 18 in step 128.

Steps 120-124 can be performed and/or initiated manually by subject 12 and/or by a user/operator. Moreover, steps 120-124 can be performed automatically in response to any triggering events/instructions, such as in response to the detection of an abnormality (or the determination that the heart sound is normal), the determination of the potential health condition, the passage of time, and/or other steps/events. Steps 120-124 can include selecting, retrieving, and/or otherwise gaining access to the information as located in storage media 22 and/or at another location, such as in the cloud as accessed via the internet. Thus, steps 120-124 can include communicating with any components of system 10 and/or any sources of information regarding subject 12, the potential health conditions, healthcare providers 18, and/or other information.

Process 100 can optionally include steps 126 and/or 128 regarding the generation and communication of a report detailing some or all information/data regarding subject 12 (e.g., report 40 as described above with regards to system 10). Steps 126 and/or 128 can be performed by, for example, report module 38 and/or communication module 42, respectively. Additionally and/or alternatively, steps 126 and/or 128 can be performed by any components of system 10 and/or any systems capable of generating and/or communicating report 40.

Step 126 can include preparing, creating, and/or otherwise generating report 40 regarding subject 12 that can include identification information of subject 12, the heart sound(s), descriptions of the heart sound(s), images representative of the heart sounds, the symptoms, the potential health condition(s), and/or any other information regarding subject 12 and/or the analysis of subject 12 for potential health conditions. Step 126 can include generating report 40 having any format and/or multiple formats (such as an audio file of the heart sounds as well as a text, word, and/or PDF file format that includes other information). Step 126 can generate report 40 manually as initiated and/or performed by a user/operator (e.g., subject 12) and/or generate report 40 automatically in response to, for example, the determination of potential health conditions and/or in response to any other triggering events/instructions. For example, step 126 can generate report 40 as instructed by subject 12 and/or in response to subject 12 making an appointment seeking assistance from healthcare provider 18. Step 126 can include accessing the information that is to be in report 40 from any components of system 10, and can be configured to provide report 40 to, for example, storage media 22 within which report 40 can be saved.

After report 40 is generated in step 126, process 100 can include step 128. Step 128 can be communicating report 40 to subject 12 and/or healthcare provider 18. Step 128 can also include providing and/or making accessible information within system 10 to components, systems, people, etc. outside (i.e., separate from) system 10. Step 128 can be performed and/or initiated manually by a user/operator (e.g., subject 12) and/or automatically in response to, for example, the generation of report 40, determination of potential health conditions, and/or any other triggering events/instructions. Step 128 can further include having access to one, multiple, or all components of system 10 (e.g., storage media 22) to access report 40 and/or the information that is to be communicated to subject 12, healthcare provider 18, and/or to any other desired parties/components. In one example, step 128 can include accessing cellular data and/or other wireless communication capabilities of mobile device 14 and/or wearable device 16 to send and receive information, such as report 40.

Process 100 can additionally and/or alternatively include step 130, which is contacting a relative, friend, and/or other individual close to subject 12, professional caregiver, and/or emergency medical personnel (such as healthcare provider 18) depending on the potential health condition. Step 130 can be performed in response to the determination that the potential health condition is serious and requires immediate medical attention. Step 130 can be performed by, for example, communication module 42 of system 10, any other components of system 10, and/or any systems capable of contacting emergency medical personnel. Step 130 can include determining that the potential health condition is serious enough to warrant contacting emergency medical personnel (which can be similar to the determination in step 122). The communication in step 130 can be via any method, including a voice call and/or a text message. Step 130 can include contacting emergency medical personnel manually as initiated and/or performed by a user/operator (e.g., subject 12) and/or automatically in response to, for example, the generation of report 40, determination of potential health conditions, and/or any other triggering events/instructions. Step 130 can include accessing one, multiple, or all components of system 10 (e.g., storage media 22) to access the information that is to be communicated to emergency medical personnel. In one example, step 130 can include using the telephone function/application on mobile device 14 to contact emergency medical personnel. In another example, step 130 can include using cellular data and/or other wireless communication capabilities of mobile device 14 and/or wearable device 16 to contact emergency medical personnel.

Process 100 can include other steps not expressly disclosed herein, such as training the machine learning model using, for example, the heart sounds, symptoms, and/or potential health conditions. The training can be performed by, for example, training module 44 as described with regards to system 10. Process 100 can include performing the disclosed steps in any order and/or multiple times as is desired and/or necessary to determine the potential health conditions and/or provide information regarding the potential health conditions.

Any of the various systems, devices, apparatuses, etc. in this disclosure can be sterilized (e.g., with heat, radiation, ethylene oxide, hydrogen peroxide, etc.) to ensure they are safe for use with subjects, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### DISCUSSION OF DETAILED EMBODIMENTS

The following are non-exclusive descriptions of possible embodiments of the present invention.

An example method of providing health condition information regarding a potential health condition of a subject is disclosed herein and can include collecting a sound of a heart of the subject; comparing the sound of the heart to a plurality of example sounds to detect an abnormality; prompting, in response to the sound having an abnormality, the providing of symptom information regarding symptoms noticeable by the subject; determining, by a first machine learning model and depending upon the sound of the heart and the symptoms of the subject, the potential health condition(s); and providing the information to the subject depending upon the potential health condition(s).

The method of the preceding paragraph can optionally include, additionally and/or alternatively, any one or more of the following features, configurations, steps, and/or additional components:

The potential health condition information can include at least one of the following: information regarding healthcare providers, a digital map showing a location of at least one healthcare provider, details about the potential health condition, information regarding a clinical trial relevant to the potential health condition, and a recommendation that the subject contacts a healthcare provider.

The step of providing the potential health condition information to the subject is performed via a user interface on an electronic mobile device.

The method can include adjusting a tolerance of the first machine learning model depending upon the symptom information.

In response to the symptom information including that the subject is experiencing no noticeable symptoms, the tolerance of the first machine learning model is focused on specificity.

In response to the symptom information including at least one symptom of the subject, the tolerance of the first machine learning model is focused on sensitivity.

The symptoms include at least one of the following: shortness of breath, chest pain, chest tightness, feeling faint, feeling dizzy, heart palpitations, difficulty moving, swelling lower extremities, difficulty sleeping, and decline in activity level.

The step of collecting the heart sound is performed by at least one of the following: an electronic mobile device that includes a microphone and an electronic wearable device that includes a microphone.

The electronic wearable device is at least one of the following: a smartwatch and a chest-worn device.

The potential health condition includes at least one of the following: atrial fibrillation, heart murmurs, structural heart valve disease, precursors to cardiac arrest, a bicuspid aortic valve, and aortic valve stenosis.

The step of comparing the heart sound to the plurality of example heart sounds to detect the abnormality is performed by the first machine learning model.

The step of comparing the heart sound to the plurality of example heart sounds to detect the abnormality further comprises: providing the sound of the heart to an abnormality detection module, accessing the plurality of example heart sounds by the abnormality detection module, and determining the abnormality depending upon the heart sound and the plurality of example heart sounds.

The method can include selecting one of the first machine learning model and a second machine learning model depending upon the symptom information, wherein the first machine learning model is configured to have higher specificity and lower sensitivity and the second machine learning model is configured to have higher sensitivity and lower specificity.

The method can include training the first machine learning model using the heart sound, the symptom information, and the potential health condition for further use with regards to the subject.

The method can include alerting the subject of a seriousness of the potential health condition.

The method can include providing a reminder notice to the subject to see a healthcare provider.

The method can include preparing a report that includes at least one of the following: the heart sound, a description of the heart sound, an image representative of the heart sound, the symptom information, and the potential health condition.

The method can include communicating, to a healthcare provider, the report.

The method can include contacting at least one of the following depending upon the potential health care condition: a relative of the subject, a friend of the subject, an individual associated with the subject, a professional caregiver of the subject, and emergency medical personnel.

The step of comparing the heart sound to example heart sounds further comprises: extracting at least one feature from the heart sound and comparing the at least one feature to example features associated with example heart sounds.

The at least one feature extracted from the heart sound includes at least one of the following: a heart sound interval, a heart sound amplitude, a heart sound frequency feature.

The example features associated with example heart sounds are stored in an example heart sound database and the method can further include accessing the example heart sound database to compare the heart sound to the example heart sounds.

The step of determining the potential health condition includes the first machine learning model determining whether the potential health condition includes the presence of a heart murmur in the subject, a second machine learning model determining whether the heart murmur is normal or abnormal, and a third machine learning model determining a severity of the potential health condition.

The above method(s) can be performed on a living animal or on a simulation, such as on a cadaver, cadaver heart, anthropomorphic ghost, simulator (e.g., with body parts, heart, tissue, etc. being simulated).

An example health monitoring and analysis system for use in providing information regarding a potential health condition of a subject is disclosed herein and can include an abnormality detection module that includes a computer processor with the abnormality detection module being configured to receive at least one heart sound of the subject, compare the heart sound to a plurality of example heart sounds, and detect an abnormality; a symptom solicitation module configured to prompt, in response to the detection of an abnormality, the subject to provide at least one symptom noticeable by the subject; a machine learning model configured to determine, depending upon the heart sound and the at least one symptom, the potential health condition; and a notification module configured to provide information to the subject depending upon the potential health condition.

The system of the preceding paragraph can optionally include, additionally and/or alternatively, any one or more of the following features, configurations, steps, and/or additional components:

The potential health condition information includes at least one of the following: information regarding healthcare providers, a digital map showing a location of at least one healthcare provider, details about the potential health condition, information regarding a clinical trial relevant to the potential health condition, and a recommendation that the subject contacts a healthcare provider.

The notification module includes a user interface that is configured to provide the health condition information to the subject.

The machine learning model is configured to adjust a tolerance depending upon the at least one symptom.

In response to the at least one symptom including that the subject is experiencing no noticeable symptoms, the tolerance of the machine learning model is focused on specificity.

In response to the at least one symptom provided by the subject including at least one symptom that is noticeable by the subject, the tolerance of the machine learning model is focused on sensitivity.

The at least one symptom includes at least one of the following: shortness of breath, chest pain, chest tightness, feeling faint, feeling dizzy, heart palpitations, difficulty moving, swelling lower extremities, difficulty sleeping, and decline in activity level.

The system can include an electronic mobile device configured to collect the heart sound.

The electronic mobile device can include a microphone.

The electronic mobile device is configured to monitor a heart of the subject to collect the heart sound.

The abnormality detection module, the symptom solicitation module, the machine learning model, and the notification module are at least partially integrated into an electronic mobile application on an electronic mobile device.

The electronic mobile device can further comprise a user interface configured to allow the subject to provide the at least one symptom.

The user interface is configured to display the information for the subject depending upon the potential health condition.

The system can include an electronic wearable device configured to collect the heart sound.

The electronic wearable device is at least one of the following: a smartwatch and a chest-worn device.

The potential health condition as determined by the machine learning model includes at least one of the following: atrial fibrillation, heart murmurs, structural heart valve disease, precursors to cardiac arrest, a bicuspid aortic valve, and aortic valve stenosis.

The system can include storage media configured to store the example heart sounds.

The storage media is configured to store the at least one heart sound of the subject.

The system can include an example heart sound database that includes the plurality of example heart sounds that is used to detect the abnormality in the at least one heart sound, wherein the abnormality detection module is configured to access the example heart sound database.

The machine learning model can further comprise: a first sub-machine learning model that is configured to have higher sensitivity and lower specificity and a second sub-machine learning model that is configured to have higher specificity and lower sensitivity.

The sub-machine learning model that is used to determine the potential health condition is selected depending on the at least one symptom.

The system can include a training module configured to train the machine learning model using the at least one heart sound, the at least one symptom, and the potential health condition.

The training module is configured to train the machine learning model for further use with regards to the subject.

The notification module is configured to provide an alert to the subject regarding a seriousness of the potential health condition.

The notification module is configured to provide a reminder notice to the subject to see a healthcare provider.

The system can include a report module configured to create a report that includes at least one of the following: a description of the at least one heart sound, an image representative of the at least one heart sound, the at least one symptom, and the potential health condition.

The system can include a communication module configured to provide the report to a specified healthcare provider.

The system can include a communication module configured to contact at least one of the following depending upon the potential health condition: a relative of the subject, a friend of the subject, an individual associated with the subject, a professional caregiver of the subject, and emergency medical personnel.

The abnormality detection module is configured to extract at least one feature from the at least one heart sound and compare the at least one feature to example features associated with the plurality of example heart sounds.

The system can include an example heart sounds database within which the example features are stored.

The at least one feature extracted from the at least one heart sound includes at least one of the following: a heart sound interval, a heart sound amplitude, a heart sound frequency feature.

The machine learning model can further comprise: a first sub-machine learning model configured to determine whether the potential health condition includes the presence of a heart murmur in the subject, a second sub-machine learning model configured to determine whether the heart murmur is normal or abnormal, and a third sub-machine learning model configured to determine a severity of the heart murmur.

The above system(s) can be used with and/or on a living animal or on a simulation, such as on a cadaver, cadaver heart, anthropomorphic ghost, simulator (e.g., with body parts, heart, tissue, etc. being simulated).

An example mobile application for use in providing information regarding a potential health condition of a subject is disclosed herein and can include a computer processor at least partially configured to receive a heart sound of the subject and perform executable software instructions to compare the heart sound to a plurality of example heart sounds; detect an abnormality in the heart sound from the comparison to the plurality of example heart sounds; prompt, in response to the heart sound having an abnormality, the subject to provide symptoms noticeable by the subject; and determine, depending upon the heart sound and the symptoms provided by the subject, the potential health condition. The example mobile application can also include a user interface configured to provide information to the subject regarding the potential health condition.

The application of the preceding paragraph can optionally include, additionally and/or alternatively, any one or more of the following features, configurations, steps, and/or additional components:

The user interface is configured to display to the subject questions regarding symptoms noticeable by the subject and collect the symptoms as provided by the subject via the user interface.

The mobile application is on/within an electronic mobile device.

The electronic mobile device includes a microphone configured to collect the heart sound of the subject.

The electronic mobile device includes storage media configured to store the executable software instructions.

The storage media is further configured to store the plurality of example heart sounds.

The potential health condition as determined by the computer processor includes at least one of the following: atrial fibrillation, heart murmurs, structural heart valve disease, precursors to cardiac arrest, a bicuspid aortic valve, and aortic valve stenosis.

The application can include an example heart sound database that includes the plurality of example heart sounds that is used to detect the abnormality in the heart sound.

The computer processor at least partially includes a machine learning model configured to determine the potential health condition.

A tolerance of the machine learning model is adjusted depending upon the symptoms provided by the subject.

In response to the symptoms provided by the subject including that the subject is experiencing no noticeable symptoms, the tolerance of the machine learning model is focused on specificity.

In response to the symptoms provided by the subject including at least one symptom that is noticeable by the subject, the tolerance of the machine learning model is focused on sensitivity.

The symptoms include at least one of the following: shortness of breath, chest pain, chest tightness, feeling faint, feeling dizzy, heart palpitations, difficulty moving, swelling lower extremities, difficulty sleeping, and decline in activity level.

The potential health condition information includes at least one of the following: information regarding healthcare providers, a digital map showing a location of at least one healthcare provider, details about the potential health condition, information regarding a clinical trial relevant to the potential health condition, and a recommendation that the subject contacts a healthcare provider.

The user interface is configured to display the information for the subject.

The computer processor includes an abnormality detection module configured to compare the heart sound to the plurality of example heart sounds and detect the abnormality therefrom.

The user interface is configured to provide an alert to the subject regarding a seriousness of the potential health condition.

The user interface is configured to provide a reminder notice to the subject to see a healthcare provider.

The computer processor is further configured to perform executable software instructions to create a report that includes at least one of the following: a description of the heart sound, an image representative of the heart sound, the symptoms, and the potential health condition.

The application can further include communication means for providing the report to a specified healthcare provider.

The computer processor is further configured to perform executable software instructions to extract at least one feature from the heart sound and compare the at least one feature to example features associated with the plurality of example heart sounds.

The at least one feature extracted from the heart sound includes at least one of the following: a heart sound interval, a heart sound amplitude, a heart sound frequency feature.

The mobile application is incorporated into a system that includes a heart sound collection device.

The heart sound collection device is sterilized.

The above system(s) and/or application(s) can be used with and/or on a living animal or on a simulation, such as on a cadaver, cadaver heart, anthropomorphic ghost, simulator (e.g., with body parts, heart, tissue, etc. being simulated).

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of providing potential health condition information regarding a potential health condition of a subject, the method comprising:
collecting a heart sound of the subject;
comparing the heart sound to a plurality of example heart sounds to detect an abnormality;
prompting, in response to the heart sound having an abnormality, a providing of symptom information regarding symptoms noticeable by the subject;
determining, by a first machine learning model and depending upon the heart sound and the symptoms of the subject, the potential health condition; and
providing the potential health condition information to the subject depending upon the potential health condition.

2. The method of claim 1, wherein the potential health condition information includes at least one of the following: information regarding healthcare providers, a digital map showing a location of at least one healthcare provider, details about the potential health condition, information regarding a clinical trial relevant to the potential health condition, and a recommendation that the subject contacts a healthcare provider.

3. The method of claim 1, wherein the step of providing the potential health condition information to the subject is performed via a user interface on an electronic mobile device.

4. The method of claim 1, further comprising:
adjusting a tolerance of the first machine learning model depending upon the symptom information.

5. The method of claim 4, wherein, in response to the symptom information including that the subject is experiencing no noticeable symptoms, the tolerance of the first machine learning model is focused on specificity.

6. The method of claim 4, wherein, in response to the symptom information including at least one symptom of the subject, the tolerance of the first machine learning model is focused on sensitivity.

7. The method of claim 1, wherein the symptoms include at least one of the following: shortness of breath, chest pain, chest tightness, feeling faint, feeling dizzy, heart palpitations, difficulty moving, swelling lower extremities, difficulty sleeping, and decline in activity level.

8. The method of claim 1, wherein the step of collecting the heart sound is performed by at least one of the following: an electronic mobile device that includes a microphone and an electronic wearable device that includes a microphone.

9. The method of claim 8, wherein the electronic wearable device is at least one of the following: a smartwatch and a chest-worn device.

10. The method of claim 1, wherein the potential health condition includes at least one of the following: atrial fibrillation, heart murmurs, structural heart valve disease, precursors to cardiac arrest, a bicuspid aortic valve, and aortic valve stenosis.

11. The method of claim 1, wherein the step of comparing the heart sound to the plurality of example heart sounds to detect the abnormality is performed by the first machine learning model.

12. The method of claim 1, wherein the step of comparing the heart sound to the plurality of example heart sounds to detect the abnormality further comprises:
providing the sound of the heart to an abnormality detection module;
accessing the plurality of example heart sounds by the abnormality detection module; and
determining the abnormality depending upon the heart sound and the plurality of example heart sounds.

13. The method of claim 1, further comprising:
selecting one of the first machine learning model and a second machine learning model depending upon the symptom information, wherein the first machine learning model is configured to have higher specificity and lower sensitivity and the second machine learning model is configured to have higher sensitivity and lower specificity.

14. The method of claim 1, further comprising:
alerting the subject of a seriousness of the potential health condition.

15. The method of claim 1, wherein comparing the heart sound to example heart sounds further comprises:
extracting at least one feature from the heart sound; and
comparing the at least one feature to example features associated with example heart sounds.

16. The method of claim 15, wherein the at least one feature extracted from the heart sound includes at least one of the following: a heart sound interval, a heart sound amplitude, a heart sound frequency feature.

17. The method of claim 1, wherein the step of determining the potential health condition includes the first machine learning model determining whether the potential health condition includes the presence of a heart murmur in the subject, a second machine learning model determining whether the heart murmur is normal or abnormal, and a third machine learning model determining a severity of the potential health condition.

18. A health monitoring and analysis system for use in providing potential health condition information regarding a potential health condition of a subject, the system comprising:
an abnormality detection module that includes a computer processor, the abnormality detection module being configured to receive at least one heart sound of the subject, compare the heart sound to a plurality of example heart sounds, and detect an abnormality;
a symptom solicitation module configured to prompt, in response to the detection of an abnormality, the subject to provide at least one symptom noticeable by the subject;
a machine learning model configured to determine, depending upon the at least one heart sound and the at least one symptom, the potential health condition; and
a notification module configured to provide the potential health condition information to the subject depending upon the potential health condition.

19. The system of claim 18, further comprising:
an example heart sound database that includes the plurality of example heart sounds that is used to detect the abnormality in the at least one heart sound,
wherein the abnormality detection module is configured to access the example heart sound database.

20. The system of claim 18, wherein the machine learning model further comprises:
a first sub-machine learning model that is configured to have higher sensitivity and lower specificity; and
a second sub-machine learning model that is configured to have higher specificity and lower sensitivity.
